(19) United States
(12) Patent Application Publication
(10) Pub. No.: US 2014/0329692 A1
(43) Pub. Date: Nov. 6, 2014

(54) METHOD FOR MEASURING DNA METHYLATION PROFILES

(71) Applicant: ALBERT EINSTEIN COLLEGE OF MEDICINE OF YESHIVA UNIVERSITY, Bronx, NY (US)

(72) Inventors: Jan Vijg, New York, NY (US); Silvia Gravina, New York, NY (US)

(73) Assignee: ALBERT EINSTEIN COLLEGE OF MEDICINE OF YESHIVA UNIVERSITY, Bronx, NY (US)

(21) Appl. No.: 14/253,922

(22) Filed: Apr. 16, 2014

Related U.S. Application Data
(60) Provisional application No. 61/817,910, filed on May 1, 2013.

(51) Int. Cl. C12Q 1/68; G01N 33/50

(52) U.S. Cl. CPC C12Q 1/6858; G01N 33/5023; C12Q 2600/154
USPC 506/2; 435/6.11; 506/9; 435/287.2

(57) ABSTRACT

Methods are provided for determining epimutations in a nucleic acid sequence of a cell.

### CROSS-REFERENCE TO RELATED APPLICATIONS

This application claims benefit of U.S. Provisional Application No. 61/817,910, filed May 1, 2013, the contents of which are hereby incorporated by reference.

### STATEMENT OF GOVERNMENT SUPPORT

This invention was made with government support under grant numbers RL1 AG032117-05 and RO1 AG034421-02 awarded by the National Institutes of Health. The government has certain rights in the invention.

### BACKGROUND OF THE INVENTION

Throughout this application various publications are referred to by number in parenthesis. Full citations for these references may be found at the end of the specification. The disclosures of these publications, and of all books, patents, and patent application publications referred to herein, are hereby incorporated by reference in their entirety into the subject application to more fully describe the art to which the subject invention pertains.

DNA methylation plays a critical role in the regulation of gene expression and is known to be an essential mechanism for guiding normal cellular development. Additionally, numerous studies have implicated aberrant methylation in the etiology of many human diseases including nearly all types of cancer (1). In the past decade, DNA methylation profiling at cytosine-guanine dinucleotides (CpG sites) has gained momentum as an epigenetic approach in basic research and clinical applications; analyses that previously were restricted to specific loci can now be performed on a genome-wide scale and entire methylomes can be characterized at single-base-pair resolution (2).

However, all techniques currently available can only measure average values obtained from cell populations as a whole, requiring at least 30 ng of DNA (i.e. the equivalent of about 6000 cells) (3). Population-wide analyses overlook individual, cell-specific changes, termed epimutations (4), and are unsuited to characterize cellular heterogeneity, which plays such an important role in differentiation and development, stem cell re-programming, diseases such as cancer, and aging (5). Developing single-cell approaches for measuring DNA methylation would not only be vital to fully understand individual cell-specific changes and complexity of tissue microenvironments, but also for the analysis of clinical samples, such as circulating tumor cells or needle biopsies, when the amount of material is often limited. Despite the recent progress in genomics and epigenomics, such methods are still lacking. The present invention addresses the need for such methods.

### SUMMARY OF THE INVENTION

This invention provides a method for determining a pattern of epimutations in a nucleic, acid sequence comprising:
optionally, mixing the nucleic acid with an amount of a heterologous nucleic acid;
subjecting the nucleic acid to nucleic acid-denaturing conditions;
contacting the nucleic acid with sodium bisulfite at a temperature in excess of 50° C. so as to convert non-methylated cytosine residues of the nucleic acid into uracil residues;
recovering the converted nucleic acid;
subjecting the recovered converted nucleic acid to amplification with primers directed to the nucleic acid sequence;
sequencing the amplified product to determine the presence of uracil residues and cytosine residues, so as to thereby determine the pattern of epimutations in the nucleic acid sequence, wherein a cytosine residue in the amplified product indicates a methylated cytosine at the corresponding residue position in the nucleic, acid sequence and wherein a uracil residue in the amplified product indicates a non-methylated cytosine at the corresponding residue position in the nucleic acid sequence.

Also provided is a method for determining the effect of an agent on epimutation status of a nucleic acid comprising determining the epimutation pattern of a nucleic acid in the cells by any of the methods described hereinabove or hereinbelow in a first portion of the cells, and contacting a second portion of the cells with the agent and determining epimutation pattern in a corresponding nucleic acid sequence in the second portion of the cells by the method of the methods described hereinabove or hereinbelow, and comparing the pattern of epimutations in the nucleic acid obtained from the first portion with the pattern of epimutations in the corresponding nucleic acid from the second portion so as to determine the effect of the agent on epimutation status of the cells.

### BRIEF DESCRIPTION OF THE DRAWINGS

FIG. 1A-1D: Schematic depiction of single-cell DNA methylation analysis (a) Single cells are lysed and treated with bisulfite to convert unmethylated cytosines into uracil. After bisulfite treatment, DNA is immediately whole-genome amplified by multiple displacement. DNA methylation patterns are analyzed in a locus-specific way using PCR amplification with primers specific for converted cytosines or genome-wide using next-generation sequencing (b) DNA methylation profile of a 180 bp fragment of the Oct4 promoter in 5-Aza-treated MEFs. Representative example of DNA methylation profile of Oct4 specific CpG site in MEFs treated with 1 μM 5-Aza for 48 and 72 hours, (c,d) DNA methylation profile of Nfe212 promoter (c) and Rabgap11 (intragenic region) (d) in single hepatocytes. Cytosines of CpG dinucleotides are highlighted in orange. Epimutations are highlighted in blue,

FIG. 2A-2E: Epimutation rates per genomic feature. Epimutation rates for each of 4 cells analyzed H1(a), H2(b), H3(c), H4(d) with demethylating and methylating events represented, respectively, by yellow and blue bars. Epimutation rates on the y-axis are calculated as percentage of epimutation sites in each genomic feature of the total number of CpG sites overlapping with the reference liver sample. (e) Circle plot made with DNAPlotter30 showing the distribution of CpG sites with methylating events (red circle) and demethylating events (green circle) of pooled samples and corresponding genomic GC content (inner circle) for chromosome 2.

### DETAILED DESCRIPTION OF THE INVENTION

This invention provides a method for determining a pattern of epimutations in a nucleic acid sequence comprising:
optionally, mixing the nucleic acid with an amount of a heterologous nucleic acid;
subjecting the nucleic acid to nucleic acid-denaturing conditions;
contacting the nucleic acid with sodium bisulfite at a temperature in excess of 50° C. so as to convert non-methylated cytosine residues of the nucleic acid into uracil residues;
recovering the converted nucleic acid;
subjecting the recovered converted nucleic acid to amplification with primers directed to the nucleic acid sequence;
sequencing the amplified product to determine the presence of uracil residues and cytosine residues, so as to thereby determine the pattern of epimutations in the nucleic acid sequence, wherein a cytosine residue in the amplified product indicates a methylated cytosine at the corresponding residue position in the nucleic acid sequence and wherein a uracil residue in the amplified product indicates a non-methylated cytosine at the corresponding residue position in the nucleic acid sequence.

In an embodiment, the method further comprises comparing the epimutation pattern of the amplified product to a corresponding control sequence, so as to determine epimutations in the nucleic acid sequence relative to the control sequence. In an embodiment, the nucleic acid is obtained from a cell.

In an embodiment, the method further comprises obtaining the nucleic acid from a single cell. In an embodiment, the heterologous nucleic acid is DNA or tRNA. In an embodiment, the heterologous nucleic acid is a salmon sperm DNA or salmon sperm tRNA. In an embodiment, the amplification primers are not directed to the heterologous nucleic acid.

In an embodiment, the nucleic acid-denaturing conditions comprise heat denaturation. In an embodiment, the nucleic acid is contacted with sodium bisulfite at a temperature of between 60° C. and 70° C. so as to convert methylated cytosine residues of the nucleic acid into uracil residues. In an embodiment, the nucleic acid is contacted with sodium bisulfite at a temperature and for an amount of time sufficient as to convert 95% or more of the methylated cytosine residues of the nucleic acid into uracil residues.

In an embodiment, the method further comprises contacting the nucleic acid, subsequent to the contact with sodium bisullite, with carrier RNA. In an embodiment, the carrier RNA is contacted with the nucleic acid in an in-column DNA purification setting.

In an embodiment, the carrier RNA comprises poly-A RNA of 100 bp to 10000 bp.

In an embodiment, the method further comprises in-column purification and a desulphonation step. In an embodiment, the desulphonation step is followed by two wash steps. In an embodiment, before the final elution, the elution buffer is warmed up to 35-39° C. In a preferred embodiment, the elution buffer is warmed up to 37° C. In an embodiment, the in-column purification is effected with a DNA-purification column.

In an embodiment, the control sequence is a whole genome sequence. In an embodiment, the control sequence is a pooled genomic sequence.

In an embodiment, the method is performed on nucleic acid obtained from a single cell. In an embodiment, the nucleic acid is mixed with the amount of the heterologous nucleic acid,

In an embodiment, the amount of the heterologous nucleic acid is 1.5 ng to 2.5 ng.

In an embodiment, the method is performed on nucleic acid obtained from plurality of cells of the same type.

In an embodiment, the recovered nucleic acid is subjected to whole genome amplification using random hexamer primers.

In an embodiment, the amplification is multiple displacement amplification. In a preferred embodiment, the standard multiple displacement amplification incubation protocol is modified so that it is performed at 15 min at 24° C., then 8 hrs 28° C.

In an embodiment, the product of whole genome amplification is a double-stranded nucleic acid. In an embodiment, the double-stranded nucleic acid is digested using a restriction endonuclease prior to sequencing. In an embodiment, the digested double-stranded nucleic acid is size-selected prior to sequencing. In an embodiment, the sequencing is massively parallel sequencing.

In an embodiment, the comparing of the sequence of the amplified product to a corresponding control sequence is conducted on a locus of the sequence. In an embodiment, the locus comprises a promoter. In an embodiment, the recovered nucleic acid is subjected to nested PCR amplification prior to sequencing.

Also provided is a method for determining the effect of an agent on epimutation status of a nucleic acid comprising determining the epimutation pattern of a nucleic acid in the cells by any of the methods described hereinabove or hereinbelow in a first portion of the cells, and contacting a second portion of the cells with the agent and determining epimutation pattern in a corresponding nucleic acid sequence in the second portion of the cells by the method of the methods described hereinabove or hereinbelow, and comparing the pattern of epimutations in the nucleic acid obtained from the first portion with the pattern of epimutations in the corresponding nucleic acid from the second portion so as to determine the effect of the agent on epimutation status of the cells.

As used herein, the term “heterologous nucleic acid” is heterologous relative to the nucleic acid for which the epimutations are being determined, i.e. a nucleic acid that is not naturally present in the cell, or in the species, from which the nucleic acid for which the epimutations are being determined has been obtained.

In an embodiment of the methods disclosed herein, the nucleic acid is obtained from a somatic cell. In an embodiment of the methods disclosed herein, the nucleic acid is obtained from a germ cell. In an embodiment of the methods disclosed herein, the nucleic acid is obtained from a eukaryote. In an embodiment of the methods disclosed herein, the nucleic acid is obtained from prokaryote. In an embodiment of the methods disclosed herein, the nucleic acid is obtained from a mammal. In an embodiment of the methods disclosed herein, the nucleic acid is obtained from a human. In an embodiment of the methods disclosed herein, the subject is a human subject and has cancer.

In an embodiment of the methods disclosed herein, the nucleic acid is obtained from a sample from a subject. In an embodiment, the sample comprises a blood sample. In an embodiment of the methods disclosed herein, the sample comprises a tissue sample. In an embodiment of the methods disclosed herein, the sample comprises a cancer cell. In an embodiment of the methods disclosed herein, the sample comprises a stem cell.

In an embodiment of the methods disclosed herein, the nucleic acid is a single cell genome.

In a preferred embodiment of the methods, the nucleic acid is DNA.

In an embodiment of the methods disclosed herein involving a restriction enzyme, the restriction enzyme is HindIII, PstI or MseI.

A system is provided for determining epimutations in nucleic acid, comprising: one or more data processing apparatus; and a computer-readable medium coupled to the one or more data processing apparatus having instructions stored thereon which, when executed by the one or more data processing apparatus, cause the one or more data processing apparatus to perform a method comprising as disclosed herein.

A computer-readable medium is provided comprising instructions stored thereon which, when executed by a data processing apparatus, causes the data processing apparatus to perform a method as disclosed herein.

As used herein, the polymerase chain reaction (“PCR”) is a technique well-known in the art to amplify a single or a few copies of a piece of DNA across several orders of magnitude by use of thermal cycling, consisting of cycles of repeated heating and cooling of the reaction for DNA melting and enzymatic replication of the DNA and primers containing sequences complementary to the target region along with a DNA polymerase (for example, see PCR Primer: A Laboratory Manual, Second Edition, edited by Carl W. Dieffenbach and Gabriela S. Dveksler, Cold Spring Harbor Laboratory Press, 2003, ISBN 978-087969654-2, which is hereby incorporated by reference).

Sequencing of a nucleic acid, as the term is used herein, can be by any method known in the art including, but not limited to, sequencing-by-synthesis methods, including chain termination methods, ligation-mediated sequencing methods, single-molecule sequencing methods, nanopore sequencing methods and semi-conductor-based sequencing methods. In embodiments the fragments are 25-50 base pairs (bp), 50-100 bp, 100-200 bp, 200-300 bp, 300-400 bp, 400-500 bp, 500-600 bp, 600-700 bp, 700-800 bp, 800-900 bp, 1000-2000 bp, 2000-3000 bp, 3000-4000 bp, 4000-5000 bp, 5000-6000 bp, 6000-7000 bp, 7000-8000 bp, 8000-9000 bp, 9000-10,000 bp, 10,000-20,000 bp, 20,000-30,000 bp, 30,000-40,000 bp, 40,000-50,000 bp, 50,000-60,000 bp, 60,000-70,000 bp, 70,000-80,000 bp, 80,000-90,000 bp, 90,000-100,000 bp, 100,000-200,000 bp, or up to 250,000 bp. Size-selection of fragments by any technique known in the art, including but not limited to agarose gel selection, can be used to select out any desired fragment size or range of fragment sizes.

In an embodiment of the methods, epimutation load at any desired locus can be derived computationally as the ratio of (sequence variants) versus (the total number of wild type sequences).

As used herein “amplifying” a given nucleic acid means increasing the copy number of that nucleic acid by, e.g., any of the standard techniques for amplifying nucleic acids known in the art.

As used herein, a “restriction enzyme” is a restriction endonuclease that cuts double-stranded or single-stranded DNA at specific recognition nucleotide sequences known as restriction sites. Restriction enzymes are well-known in the art. In an embodiment, the restriction enzyme is a 4-base cutter. In an embodiment, the restriction enzyme is HindIII, PstI or MseI.

In an embodiment, the sequencing performed is performed by Sanger technique. In an embodiment, the sequencing is a massively parallel sequencing technique.

As used herein a “reference nucleic acid sequence” is a nucleic acid sequence which is used as a standard for mapping and comparing other sequences to, for purposes of identifying differences. For example, a reference nucleic acid sequence, usually predetermined, may be obtained from a database available in the art, e.g. RefSeq as supplied at www.ncbi.nlm.nih.gov/RefSeq/, or obtained by sequencing a nucleic acid from, for example, other members, including a plurality of, the cell, tissue or subject population on which the method is being applied. In one embodiment, the reference sequence is the human genome hg19. In an embodiment, the reference nucleic acid sequence is the wildtype nucleic acid sequence.

As used herein a “corresponding portion” of a reference nucleic sequence is a portion of the reference nucleic sequence that aligns with or matches, as determined for example by sequence alignment/map tools widely available in the art, the sequence being compared.

The epimutation status is determined by the methods described. Because the bisulfite conversion of the tested nucleic acid results in unmethylated cytosines being turned into uracil residues, comparing the sequence of the resultant converted nucleic acid to a reference sequence, for example a wildtype sequence, will inform one which cytosines were methylated in the tested nucleic acid. For example, a uracil present in the converted nucleic acid at a residue position corresponding to a cytosine residue in the reference sequence, indicates that that cytosine is unmethylated in the tested nucleic acid. For example, a cytosine residue present in the converted nucleic acid at a residue position corresponding to a cytosine residue in the reference sequence, indicates that that cytosine is methylated in the tested nucleic acid, and if the corresponding cytosine residue is not indicated as methylated in the reference sequence, then a difference is demonstrated. A pattern of the epimutations can be constructed, and can be compared to the pattern of the epimutations in the reference sequence. An epimutation as used herein refers to the methylation or demethylation of a DNA residue, typically a cytosine at a CpG, relative to the non-epimutated equivalent. Thus, an epimutation as used herein can be a dmethylation at a residue normally methylated, or a methylation at a residue normally demethylated (e.g. as compared to a control sequence or wild-type sequence).

In an embodiment, the amplification employed is multiple displacement amplification. In an embodiment, the amplification uses a pHi29 DNA polymerase. In an embodiment, the amplification is isothermal.

In an embodiment, recovering the converted nucleic acid comprises purifying the converted nucleic acid from remaining reagents and products using a nucleic acid purification column.

In an embodiment, the method further comprises desulphonating the recovered nucleic acid.

In an embodiment, 95% or more of the methylated cytosine residues in the nucleic acid sequence are converted into uracil residues. In an embodiment, 96% or more of the methylated cytosine residues in the nucleic acid sequence are converted into uracil residues. In an embodiment, 97% or more of the methylated cytosine residues in the nucleic acid sequence are converted into uracil residues. In an embodiment, 98% or more of the methylated cytosine residues in the nucleic acid sequence are converted into uracil residues. In an embodiment, 99% or more of the methylated cytosine residues in the nucleic acid sequence are converted into uracil residues. In an embodiment, 99.4% or more of the methylated cytosine residues in the nucleic acid sequence are converted into uracil residues.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network, Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

Accordingly, a system is provided for performing the methods as described herein comprising: one or more data processing apparatus; and a computer-readable medium coupled to the one or more data processing apparatus having instructions stored thereon which, when executed by the one or more data processing apparatus, cause the one or more data processing apparatus to perform the methods as described herein.

Also provided is a computer-readable medium coupled to the one or more data processing apparatus having instructions stored thereon which, when executed by the one or more data processing apparatus, cause the one or more data processing apparatus to perform a method as described herein.

Where a numerical range is provided herein, it is understood that all numerical subsets of that range, and all the individual integers contained therein, are provided as part of the invention. Thus, a fragment which is, for example, from 25 to 50 base pairs in length includes the subset of fragments which are 25 to 30 base pairs in length, the subset of fragments which are 35 to 42 base pairs in length etc. as well as a fragment which is 25 base pairs in length, a fragment which is 26 base pairs in length, a fragment which is 27 base pairs in length, etc. up to and including a fragment which is 50 base pairs in length.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

Stochastic epigenetic changes drive biological processes, such as development, aging and disease. Yet, epigenetic information is typically collected from millions of cells, thereby precluding a more precise understanding of cell-to-cell variability and the pathogenic history of epimutations. Herein is presented a novel procedure for DNA methyl-typing, which in an embodiment involves detection of DNA methylation at the base pair level in single cells, within promoter regions of genes or in a genome-wide manner, using whole genome amplification coupled with bisulfite sequencing. By comparing genome-wide DNA methylation patterns between single mouse hepatocytes and total genomic liver DNA we show epimutation rates 2-3 orders of magnitude higher than mutation rates. Demethylating epimutations appeared to be enriched particularly in gene bodies and repeat sequences, while methylating epimutations are homogenously distributed across the genome.

Methods to accurately detect DNA methylation at specific loci typically involve treating DNA with sodium bisulfite (6) or digesting DNA with cytosine methylation-dependent endonucleases (7). The latter approach is incapable of analyzing all possible methylation sites. Moreover, restriction digestion of single-cell genomes is impractical and highly unlikely to come even close to being complete. By contrast, bisulfite conversion of unmethylated cytosines into uracil is a relatively simple chemical reaction, which has now become a standard in DNA methylation profiling (8). A key advantage of this method is accuracy, as the degree of methylation at each cytosine can be quantified with great precision.

### Results

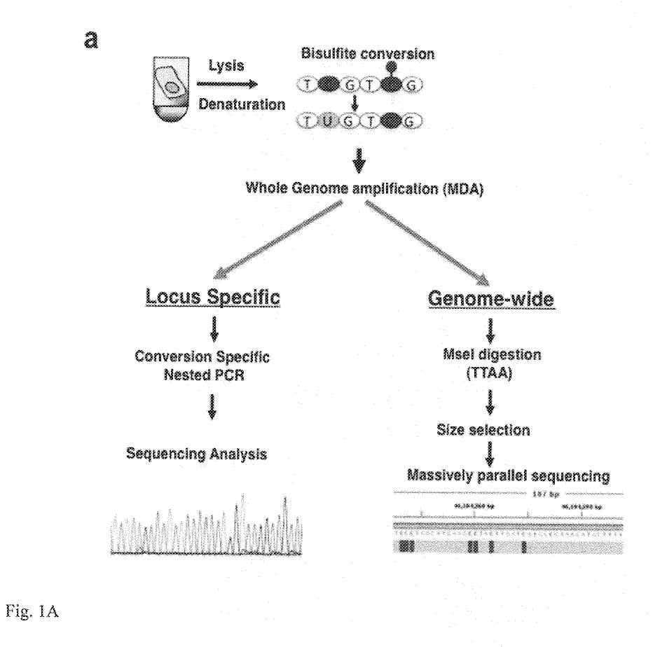

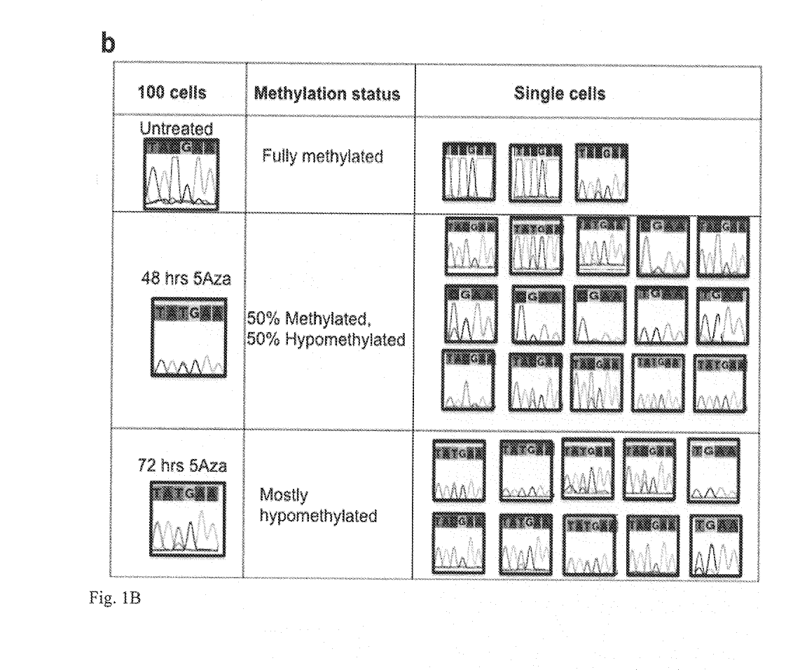

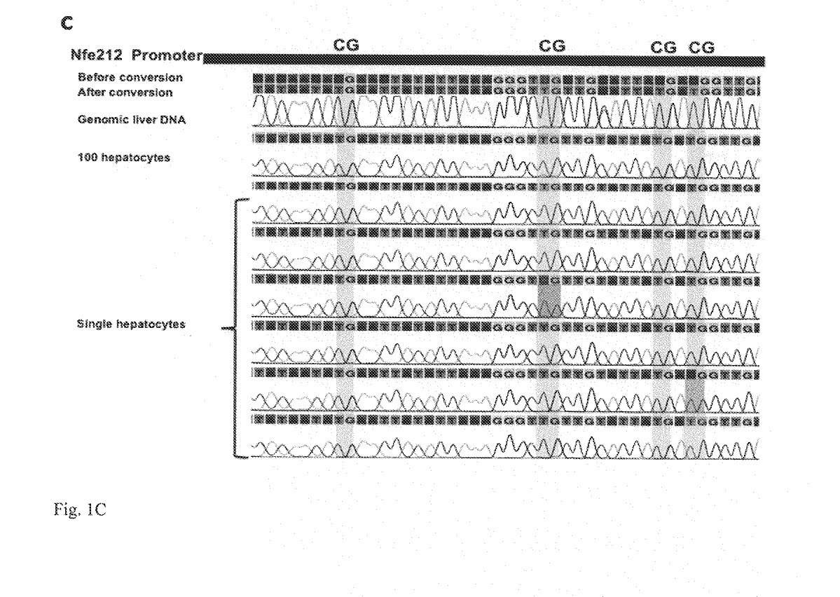

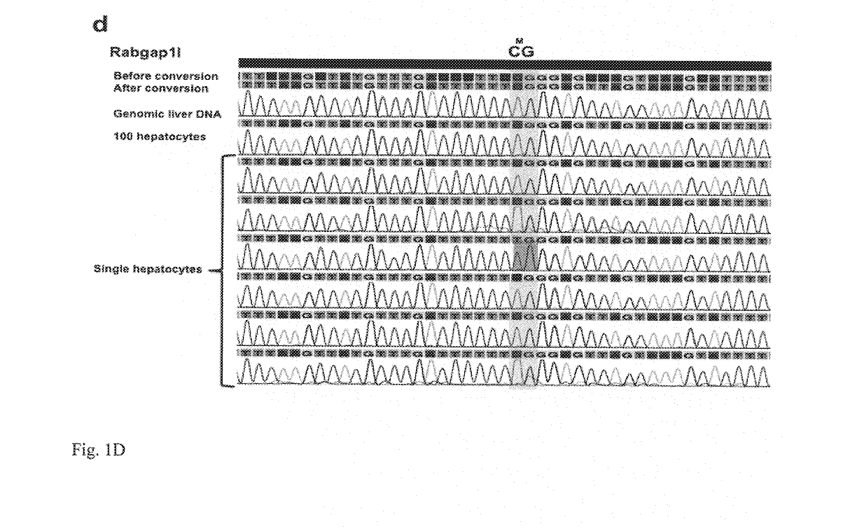

A novel method for DNA methylation analysis of single cells disclosed here uses a combination of optimized bisulfite treatment and whole genome amplification. Single cells were collected from populations of mouse embryonic fibroblasts (MEFs) or hepatocytes, under an inverted microscope by hand-held capillaries, and either frozen or immediately subjected to heat DNA denaturation, followed by bisulfite treatment. The converted DNA was subsequently subjected to whole genome amplification using multiple displacement amplification (MDA), based on phi29 DNA polymerase and random hexamer primers in an isothermal reaction. The bisulfite-converted, amplified material was then used as template for conversion-specific PCR, targeting regions of interests, or to make reduced-representation libraries (see below) for Next Generation Sequencing (NGS). The procedure is schematically depicted in FIG. 1A.

One of the major limitations of bisulfite sequencing for DNA methylation analysis is its propensity to DNA degradation due to partial, acid-catalyzed depurination (9). Consequently a high proportion of the template DNA is too fragmented to be analyzed. In addition, if the treatment is too harsh or prolonged, a small portion of 5-methylcytosines may be converted to uracil, resulting in falsely concluding the absence of methylation (10). Milder treatment, conversely, results in incomplete conversion and false positives.

Therefore, in this protocol, a balance between under-treatment and over-treatment has to be obtained, trying to achieve both a high sensitivity and high specificity in detecting epimutations. To accomplish this, bisulfite conversions were investigated by testing promoter regions of genes known to be either hyper-methylated or hypo-methylated in MEFs, i.e., 141 bp of the promoter region of the Nfe212 stress response gene, which is constitutively expressed and hypomethylated in MEFs (11), and 180 bp of the promoter region of the transcription factor Oct4 (also known as Pou5f1), which is both methylated and non-expressed in differentiated cells (12). PCR primers were designed to amplify only converted sequences, that is, sequences in which non-methylated cytosines are replaced by thymines. To increase specificity, a nested PCR approach was used. As positive controls, collections of 100 MEFs, bisulfite-treated were used and MDA-amplified in the same way as the single cells, as well as 800 ng of bisulfite-treated unamplified DNA from the same MEF population. Non-bisulfite-treated, non-amplified, genomic DNA served as negative control to verify PCR specificity for only fully converted DNA.

Using these novel approaches, initial results indicated that bisulfite conversion of cytosines at relatively low temperatures 37° C.) is generally incomplete (˜80% conversion rate; data not shown). An optimal degradation versus conversion ratio was obtained by incubating DNA for 3.5 hours at 64° C. Under these conditions, full conversion of unmethylated cytosines was obtained with minimal degradation. However, occasional conversion of methylated CpGs in the single cells was also observed but not in the controls.

It was hypothesized that this was possibly due to the bisulfite treatment conditions being too harsh for the only ˜5 pg of DNA in a single cell, but not for the 100-cell sample or 800 ng total genomic DNA. Therefore, an additional protocol step was performed by adding 2 ng (i.e. the equivalent of a few hundred cells) of salmon sperm DNA or tRNA as a “competitor” to the single cell DNA in the conversion reaction in order to reduce the over-exposure of the single cell DNA to the bisulfite. Under these conditions it was possible to greatly reduce conversion of methylated cytosines while maintaining high conversion of unmethylated cytosines. With the newly modified protocol, nested PCR for the Nfe212 promoter resulted in a product in about 40% and 99% of the time in single cells and 100-cell samples, respectively; similar results were obtained for Oct4 (not shown).

It was concluded that DNA degradation by the bisulfite treatment is most likely responsible for the less than 100% success rate in obtaining a correct PCR fragment after conversion. This is difficult to ascertain because MDA frequently fails in whole genome amplification of single cells and it is unclear if this is due to the simple absence of a single cell where there should be one or other, unknown factors. At this stage it was considered that the protocol was able to determine DNA methylation in gene promoter regions at great accuracy in single cells, which is demonstrated by the data below. Of note, conversion-specific PCR obviously enriches for highly converted parts of the genome and these results do not rule out the possibility of incomplete conversion elsewhere in the same genome (see below).

To definitively test the protocol, it was investigated whether it was capable of directly detecting epimutations, i.e., random changes in methylation status of single cytosines as a consequence of, for example, errors in de novo or maintenance methylation. While estimates of DNA methylation accuracy have been reported (13, 14), it has never been possible to directly determine such epimutations. To test if the method was able to detect single cell-specific epimutations, a time course experiment was performed in MEFs treated with 5-azacytidine (5-Aza). 5-Aza prevents methylation at CpG sites in newly synthesized DNA through covalent binding to Dnmt1 (15). It has been shown that 5-Aza treatment leads to a significant decrease in global methylation (16). Cultured cells were treated with 1 μM 5-Aza for 48 and 72 hours; ten single cells as well as 100-cell controls from each treatment group were subjected to lysis and bisulfite treatment followed by MDA amplification. Then the promoter region of Oct4 was tested and it was shown that a fully methylated pattern of CpG sites was present in the untreated single fibroblasts as well as in the 100-cell control (FIG. 1B, upper panel). After 72 hours of treatment the Oct4 promoter was fully demethylated, both in single fibroblasts and in the 100-cell control sample (FIG. 1B, lower panel). After 48 hours of treatment, DNA demethylation was incomplete as could be concluded from the 100-cell sample, which showed <50% demethylation. This suggested the presence of a mixed population of unmethylated and methylated CpG sites. Indeed, when the Oct4 promoter was analyzed in single fibroblasts upon 48 hours of treatment, a mixed population was found comprising either methylated or unmethylated cytosines (FIG. 1B, middle panel),

The protocol was investigated for testing epimutation rates by analyzing DNA methylation in single hepatocytes isolated from different mice. As targets, regions of genes were selected that either are constitutively expressed and hypomethylated, or repressed and hypermethylated in liver. Examples of a number of single hepatocytes for two genes, Nfe212 and Rabgap11, are shown in FIGS. 1C and 1D. Table 1 summarizes the results obtained with a total of 601 CpG sites interrogated.

TABLE 1
Locus-specific bisulfite sequencing of single hepatocytes.

| | Number | | Total CpGs | | | Conversion of |
|---|---|---|---|---|---|---|
| | of CpGs | Total | for all | | | unmethylated |
| | per | cells | the cells | Total epimutations | | non-CpGs |
| Gene region | region | analyzed | analyzed | Demethylating | Methylating | cytosines |
| Oct4 (region1) | 2 | 17 | 34 | 6 | | 290/293 |
| Oct 4 (region 2) | 4 | 12 | 48 | 3 | | 230/230 |
| L1 (Chr18) | 2 | 33 | 66 | 3 | | 48/48 |
| Gabra-1 | 3 | 66 | 198 | | | 724/726 |
| Cyp71a | 2 | 24 | 48 | | 4 | 733/740 |
| Nfe212 | 4 | 34 | 136 | | 6 | 428/429 |
| Rabgap1l | 4 | 11 | 44 | 3 | | 692/695 |
| Dpf1 | 3 | 9 | 27 | 1 | | 151/152 |
| | | | 601 | 16 | 10 | 3279/3296 |
| | | | | (Demethylating | (Methylating | (Conversion |
| | | | | epimutation | epimutation | rate: 99.4%) |
| | | | | rate: 2.6%) | rate: 1.6%) | |

In non-pluripotent cell types, cytosines not followed by guanines are methylated only very rarely (17), and this was used as an indication of bisulfite conversion efficiency, calculating the C to T conversion rate for all cytosine bases other than those in CpG dinucleotides. Out of a total of 3296 non-CpG cytosines, 3279 were converted into uracil (and subsequently thymine), a rate of well over 99.4%. To identify differential methylation and demethylation events (hereafter called epimutations), the single-cell DNA methylation patterns were compared with the liver tissue methylation patterns. Epimutation rates were estimated as 2.6% and 1.6% for demethylating and methylating events, respectively (Table 1). Of note, because the cells studied are diploid (or may be even polyploid in the case of hepatocytes) (18), at least two alleles per cell are targeted. However, MDA has allelic bias, and sometimes amplification occurs from one allele only (19). Such amplification bias is randomly distributed across the genome. Therefore, it cannot be ruled out that a single peak in the Sanger sequence truly reflects homozygous methylation status or is due to allelic bias of the amplification. Therefore, in calculating epimutation rates, unless two peaks could clearly be distinguished in the Sanger sequence, the assumption was made that only one allele is represented. In addition, while for the demethylating epimutations the possibility that these represent accidental conversions of methylated cytosines rather than genuine demethylating events cannot be ruled out, for the methylation mutations it was verified that their rate was significantly higher than the non-conversion rate of unmethylated cytosines (Fisher's exact test, p<0.05).

Thus it was demonstrated that the protocol is capable of determining DNA methylation status of gene promoter regions using conversion-specific PCR and Sanger sequencing. To demonstrate that the protocol can be applied equally effective to the genome overall, eventually resolving the entire, single-cell methylome, next-generation sequencing was used. For this purpose a reduced representation approach was adopted (Reduced Representation Bisulfite Sequencing; RRBS) (20), which reduces costs (FIG. 1A). Four single mouse hepatocytes (H1, H2, H3 and H4) were denatured, bisulfite-converted and MDA-amplified according to our protocol. In parallel, 200 ng of total genomic liver DNA was subjected to the same procedure. Amplification products were then digested with the restriction enzyme MseI, which cuts at TTAA sites. After digestion, a size selection (250-300 bp) was performed, which limited our analysis to ˜10% of the genome. An in silico digestion of the converted mouse genome indicated that this size range would allow us to interrogate ˜1.2 million CpG sites. The size-selected DNAs from the 4 single cell samples and the liver sample were end-repaired, A-tailed and ligated to Illumina adapters, and the completed libraries were sequenced to 120 bp on the Illumina HiSeq 2000.

Preprocessed and mapping of all samples was performed as described hereinbelow. Table 2 summarizes the mapping statistics for all samples. As expected, the coverage in single cell samples was lower than in the liver sample, most likely due to degradation by the bisulfite and uneven amplification.

TABLE 2
General statistics of RRBS of 4 hepatocytes

| | | Bisulfite | | | Unique | Unique CpGs |
|---|---|---|---|---|---|---|
| | | conversion | Total | Mapped | CpG | covered by |
| Sample | Source | rate | reads | reads | positions | >= 2 reads |
| H1 | single | 48.86% | 156,927,278 | 18,271,029 | 3,475,195 | 521,179 |
| | hepatocyte | | | | | |
| H2 | single | 58.78% | 144,431,273 | 13,332,253 | 2,735,879 | 382,005 |
| | hepatocyte | | | | | |
| H3 | single | 55.75% | 188,656,322 | 30,189,174 | 2,067,484 | 301,791 |
| | hepatocyte | | | | | |
| H4 | single | 60.96% | 110,330,731 | 7,405,567 | 1,375,123 | 216,244 |
| | hepatocyte | | | | | |
| Liver | genomic | 79.96% | 85,867,213 | 18,454,394 | 3,349,890 | 1,155,708 |
| | liver DNA | | | | | |

As mentioned above, non-CpG methylation is rare and its detection in any given read has been treated as an indication of inadequate bisulfite treatment. In previous work by others (21) only reads exhibiting zero non-CpG methylation were included in further analysis (i.e. selecting for highly converted reads). This is similar to what was done in the aforementioned locus specific assay, in which highly converted DNA was automatically selected for by designing primers to target only fully converted material. However, in single-cell analysis with its relatively high degradation and therefore low coverage, selecting only for highly converted regions removed over 90% of all mappable reads. This would leave one with too few reads for a robust analysis. Moreover, the selection of reads with high non-CpG converion did not seem to guarantee a high conversion rate of non methylated CpGs).

To address this issue a more sophisticated statistical model was used which accounts for over-conversion and non-conversion in the respective samples, thus allowing exploitation of the information contained in the vast amount of incompletely converted DNA. In a Bayesian approach, which models the methylation levels not at a single nucleotide level but in a region of fixed size, prior knowledge about the general CpG methylation levels in such regions was combined with the experimental data at hand. Since per-position methylation rates in cell mixtures typically range between 10% and 90%, there must inevitably be a high cell-to-cell variance of methylation at the single position level (22); therefore, the identification of regional changes in methylation levels is functionally more relevant and statistically more robust. Indeed, by comparing single position versus region-based epimutation calling, it was possible to show a reduction in false positive and false negative methylation calling rates.

By employing a method for calling regional epimutation events, the single-cell (H1, H2, H3, H4) DNA methylation patterns were compared with that of the liver tissue as reference. Epimutation rates were 1.9%-4.7% for demethylating events and 0.2%-1% for methylating events (Table 3).

TABLE 3
Results from methylation analysis per
sample, all reads, model-adjusted

| | | | | Meth- | Demeth- |
|---|---|---|---|---|---|
| | Overlapping CpG | Fully | Fully | ylating | ylating |
| Sam- | positions with | meth- | unmeth- | epimutations | epimutations |
| ple | liver tissue | ylated | ylated | (%) | (%) |
| H1 | 12,635 | 57 | 12,268 | 27 (0.21) | 596 (4.71) |
| H2 | 11,333 | 162 | 10,682 | 84 (0.74) | 431 (3.83) |
| H3 | 7,417 | 150 | 6,808 | 81 (1.09) | 186 (2.50) |
| H4 | 4,150 | 28 | 4,034 | 14 (0.33) | 79 (1.90) |

All CpG positions with a minimum coverage of 2 reads were analyzed and epimutations were called. The table shows the positions in common with the liver tissue, which serves as reference, the fraction of fully methylated and unmethylated positions and the epimutation sites and rates. Epimutation rates are defined as the ratio of epimutation sites to the total common positions between each single cell and the reference sample.

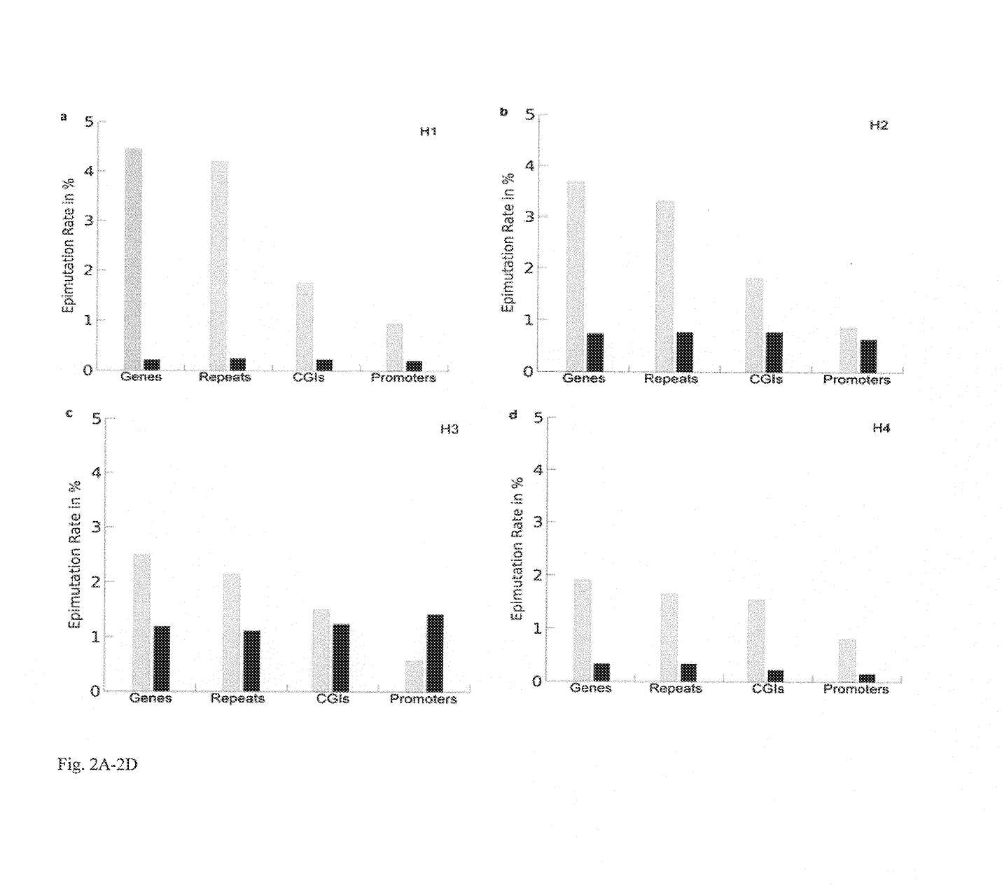

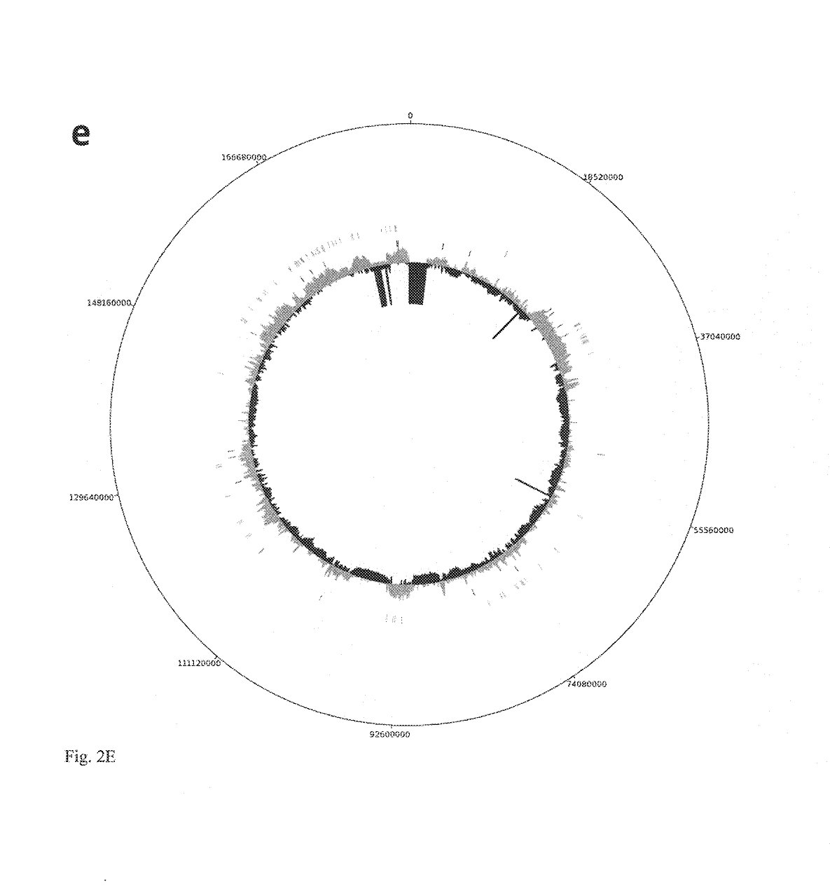

As we could also deduce from the Sanger sequencing of gene promoter regions, demethylating events were more common than methylating events, perhaps due to errors generated by DNMT1 in the propagation of methylation patterns during cell division. The distribution of genomic features across epimutation-associated sites is illustrated in FIG. 2A,B,C,D with epimutation site distribution for a representative chromosome (chromosome 2) shown in FIG. 2E. Counts of genomic features per sample are reported in Supplementary Table 3. Interestingly, the analysis reveals demethylating epimutations being enriched in gene bodies and repeat regions while promoter regions are much more stable than the rest of the genome. Conversely, methylating epimutations are homogenously distributed across the genome.

This high propensity for repeats and gene bodies to accumulate demethylating epimutations could arise because such genomic features are highly methylated and therefore more prone to accumulate demethylating epitmutations. Such events could occur as errors in the activity of DNMT1 in propagating DNA methylation patterns. This finding is in accordance with prior observations of mammalian methylomes in aging, which found that most aberrant changes in coding regions consist of demethylation, with aberrant methylation occurring mostly in CGIs and promoters (23). As expected, epimutation events are distributed heterogeneously among chromosomes, pointing toward a stochastic mechanism, similar to DNA sequence mutations (data not shown). While only four cells were analyzed, these results also indicate considerable cell-to-cell variation.

This is the first procedure that is able to accurately analyze DNA methylation patterns at a single-cell resolution. The main new insight that can be derived from our analyses is that at 0.2-4.7% DNA epimutation rate is 2-3 orders of magnitude higher than the DNA sequence mutation rate (24). This finding is in accordance with recent results suggesting that spontaneous transgenerational epigenetic changes in the Arabidopsis thaliana methylome are three orders of magnitude more frequent than DNA mutations (25), (26). The high propensity of accumulating demethylating epimutations, particularly in repetitive elements, is in keeping with recent findings suggesting an age-dependent global hypomethylation of transposed elements such as Alu elements (27, 28).

The single-cell bisulfite reduced representation approach can be applied not only to basic research on phenotypic diversity within organs and tissues in relation to disease states, but also to improve diagnostic and prognostic assays that sample very small numbers of cells from affected areas of diseased tissues. One major clinical application is to assess DNA methylation patterns in promoter regions of tumor suppressor genes in circulating tumor cells (29).

It has been well documented that bisulfite degrades DNA; approximately 84-96% of the DNA is lost during standard procedures (32, 33). Therefore, it is assumed that very small amounts of DNA, such as in a single cell, would be degraded too, which essentially constrains the possibility to perform DNA methylation analysis of single cells under standard conditions.

Indeed, while one would expect that improvements of conditions could lead to a protocol for DNA methylation analysis is small groups of cells, e.g., hundred to several hundred, those ordinary skills in the art might consider DNA methylation of single cells through bisulfite sequencing under standard conditions impossible. Hence, the observation herein that, after technical innovations such as:
addition of Salmon Sperm DNA before bisulfite conversion to buffer DNA degradation;
addition of carrier RNA in the in-column DNA purification (which enhances the recovery of DNA by preventing the small amount of target nucleic acid present in the sample from being irretrievably bound);
the incubation of the elution buffer at 37° C. (before the final elution step);
the introduction of a whole genome amplification step (MDA) upon bisulfite conversion; and
the introduction of modifications to standard MDA incubation protocol (1.5 min at 24° C., 8 hrs at 28° C.),
DNA methylation analysis through bisulfite sequencing is actually feasible, was not expected.

Having access to such sensitive technology can shift emphasis from average endpoints towards the description of cell populations, tissues and organs through their individual parts at single-cell resolution.

### Methods and Materials

Animals and tissue collection: Young (6 months) and old (27 months) C57BL/6 mice were obtained from the National Institute on Aging (NIA). All surgical procedures and experimental manipulations were approved by the Ethics Committee for Animal Experiments at the Albert Einstein College of Medicine. Experiments were conducted under the control of the Guidelines for Animal Experimentation. Animals were sacrificed by cervical dislocation.

Isolation of single mouse embryonic fibroblasts: Mouse embryonic fibroblasts (MEFs) were isolated from embryonic day 13.5 embryos of C57BL/6 mice as described (31). All cultures were maintained in a 3% O₂ and 5% CO₂ atmosphere. The solutions of 5-aza-2′-deoxycytidine (5-Aza; Sigma, St. Louis, Mo., USA) in culturing medium were prepared at the time of application and applied every 24 hours for 72 hours. After trypsinization, single MEFs were collected under an inverted microscope by hand-held capillaries, deposited in PCR tubes and immediately frozen on dry ice and stored at 80° C. until needed or immediately bisulfite-converted.

Isolation of single hepatocytes: Mouse liver tissue was perfused with collagenase following the protocol as described (18). Single hepatocytes were collected under an inverted microscope as described for MEFs.

Genomic DNA extraction: DNA from MEF cultures and liver from C57BL/6 mouse was isolated by phenol/chloroform extraction, as described (24).

Bisulfite conversion: The bisulfite conversion and recovery of DNA were performed using the EZ DNA Methylation-Direct Kit (Zymo Research, Orange, Calif.). Bisulfite conversion was performed according to the instructions of the supplier with some modifications. First the bisulfite solution was added to the single or 100 cells together with 2 ng of salmon sperm DNA or tRNA. DNA was denaturated for 8 min at 99° C. and immediately bisulfite-modified at 64° C. for 3.5 hours. After conversion, 6 μl of (4μg/μl) carrier RNA (Qiagen) was added to the DNA binding buffer solution. The addition of carrier RNA enhances the recovery of DNA by preventing the small amount of target nucleic acid present in the sample from being irretrievably bound. Converted DNA was then added to the binding solution/carrier RNA solution and subjected to an in-column purification and desulphonation step followed by two wash steps. Before the final elution, the elution buffer was warmed up at 37° C. and then allowed to sit on the column for a few minutes. DNA was eluted in 10 μl of buffer. This protocol was applied to single cells, 100 cell samples and genomic DNA extracted from the mother population. Immediately upon conversion, converted single-cell or 100-cell DNA was subjected to whole genome amplification. Genomic DNA used as control (starting material 800 ng) employed for the locus specific assay was not subjected to whole genome amplification. Note that for the RRBS, the converted DNA (starting material 200 ng) was instead subjected to whole genome amplification in order to generate a double stranded DNA (see below for details).

Whole genome amplification: The whole genome amplification of the bisulfite-treated DNA was done using a multiple displacement amplification (MDA). The amplification steps were performed using the Whole Bisulfitome kit (Qiagen) according to the instructions of the supplier with some modifications. Master mix and Phi29 DNA polymerase were added to the eluted single cell and 100 cell bisulfite-treated DNA and incubated as follow: 15 mm at 24° C., 16 hrs at 28° C. followed by a polymerase inactivation step at 95° C. for 5 minutes. For the RRBS approach, we additionally amplified the bisulfite converted DNA (starting material was 200 ng) as follows: 15 min at 24° C., 8 hrs at 28° C. followed by a polymerase inactivation step at 95° C. for 5 minutes.

Bisulfite conversion-specific PCR: A nested PCR was performed to amplify the target regions. Primers were designed using Sequenom's EpiDesigner software and synthesized by DT (Coralville, Iowa, USA). Sequences of the primers are available in the primer list (Table 4).

TABLE 4
Primers used.

| Gene name | Primer sequence |
|---|---|
| Nfe212FE | 5′-AGGAAGAGAGTGGTATAGTTTTTAGTTTGTGGAGAGT -3′ |
| Nfe212RE | 5′-CAGTAATACGACTCACTATAGGGAGAAGGCTCCACCCAAAACCCAACAAAC-3′ |
| Nfe212FI | 5′-AGGAAGAGAGAGTTATGAAGTAGTAGTAAAAA-3′ |
| Nfe212RI | 5′-CAGTAATACGACTCACTATAGGGAGAAGGCTAATATAATCTCATAAAACCCCAC-3′ |
| Cyp7taFE | 5′-GAGTGAATTTTTAAGTTATGGTTGTTT-3′ |
| Cyp7taRE | 5′-AACAAACAAAAACTTTCCATCCTAA-3′ |
| Cyp7taFI | 5′-AGGAAGAGAGTTGTTTAGAAGATGAGTGTTGGGAG-3′ |
| Cyp7taRI | 5′-CAGTAATACGACTCACTATAGGGAGAAGGCTCAAACAAAAACTTTCCATCCTAA |
| | CTT-3′ |
| RabFE | 5′-AGGAAGAGAGTGATTGGGAGTTTTGTAGTTTTGTA-3′ |
| RabRE | 5′-CAGTAATACGACTCACTATAGGGAGAAGGCTAAAAAAAACAATTACTCTAACTTTCA |
| | CA-3′ |
| RabFI | 5′-AGGAAGAGAGGTGGTAAAGTGTTTGTTTATAGAAGGG-3′ |
| RabRI | 5′-CAGTAATACGACTCACTATAGGGAGAAGGCTAAATAAACCTCCAAATCTACTACCC-3′ |
| OCT4-reg2-FE | 5′-GTTTTGGATATGGGTTGAAATATTG-3′ |
| OCT4-reg2-RE | 5′-CCACCCTCTAACCTTAACCTCTAAC-3′ |
| OCT4-reg2-FI | 5′-AGGAAGAGAGGATATGGGTTGAAATATTGGGTTTAT-3′ |
| OCT4-reg2-RI | 5′-CAGTAATACGACTCACTATAGGGAGAAGGCTAATCCTCTCACCCCTACCTTAAAT-3′ |
| OCT4-reg1-FE | 5′-AGGAAGAGAGGAAGGTTGAAAATGAAGGTTTTTT-3′ |
| OCT4-reg1- | 5′-CAGTAATACGACTCACTATAGGGAGAAGGCTCCACCCTCTAACCTTAACCTCTAAC-3′ |
| RE & RI | |
| OCT4-reg1-FI | 5′-AGGAAGAGAGAAGGTTGAAAATGAAGGTTTTTT G-3′ |
| L1chr18FE | 5′-TATAGGGGAATGTTAGGGTTAAGAAG-3′ |
| L1chr18RE | 5′-CAA AAC AAT ACC ACC TCA AAA CTA CT-3′ |
| L1chr18FI | 5′-AGGAAGAGAGATAGGGGAATGTTAGGGTTAAGAAG-3′ |
| L1chr18RI | 5′-CAGTAATACGACTCACTATAGGGAGAAGGCTCCTAAAACCTAAAAATTACAAAC |
| | ATAATT-3′ |
| Dpf1FE | 5′-TTTTTGGGATTTAGTTTTTGTTTTA-3′ |
| Dpf1RE | 5′-CAGTAATACGACTCACTATAGGGAGAAGGCTACAAAAACCTTTTCCTTCAAATA |
| | CC-3′ |
| Dpf1FI | 5′-AGGAAGAGAGAGGATTAGTTGTTGTTTTGTGATGA-3′ |
| Gabra1FE | 5′-TTTTTAGGGAAGGTAAGAAAAGAGGAT-3′ |
| Gabra1RE & | 5′-CAACATAAAAAACAACCAAACAAAC-3′ |
| RI | |
| Gabra1FI | 5′-AGGAAGAGAGTGGAGTTTAAGGTAAAGGTATGTTTT-3′ |

A T7-promoter tag was added to the reverse, primer, and a 10-mer-tag sequence was added to the forward primer to balance the PCR primer length. Approximately 5 μl of modified DNA was used as a template in the first round of PCR amplification. The reaction was carried out using HotStarTaq Master Mix (Qiagen) in a total of 50 μl reaction as follows: an initial heat-activation step at 95° C. for 15 min, 95° C. for 30 sec, annealing for 1 min, 72° C. for 1 min for a total of 30 cycles ending with a final extension at 72° C. for 10 min. The nested PCRs were performed on 5 μl of the first round PCR products. The PCR products were purified using a QIAquick PCR purification kit (Qiagen). Amplicons were Sanger sequenced on the ABI 3730 DNA sequencer in the Albert Einstein Genomic Core Facility.

RRBS and library preparation: Up to 2 μg of bisulfite-treated and whole genome amplified DNA from a single hepatocyte or mother population was purified using AMPure XP magnetic beads (Agencourt, Beverly, Mass.). DNA was digested with 50 U of MseI (NEB, Ipswich, Mass.), and size-selected using 1.5% agarose to 250-350 bp. The size-selected material was end-repaired and A-tailed and ligated to Illumina paired-end adapters. Approximately 10 ng of the product was used as input for 10 cycles of enrichment PCR with 2 U of Platinum Pfx DNA polymerase (Invitrogen, Carlsbad, Calif.), 1× Pfx buffer, 2 mM MgSO4, 400 μM dNTPs, and 1 μM Illumina enrichment PCR primers (IDT, Coralville, Iowa). The completed libraries were sequenced to 120 bp on the Illumina HiSeq 2000. All lanes were spiked with bacteriophage PhiX174 (30%) as standard quality control and to obtain a balanced representation of bases at the beginning of each read.

Preprocessing, mapping and postprocessing of reads: FastQ data was first subjected to quality control by FastQC 0.10 (34) to identify overrepresented sequences and low-quality ends. Several overrepresented sequences per sample were identified. Foreign sequence at the start or end of a read was treated as adapter contamination and clipped from the ends of all reads using Cutadapt 1.0 (35). Using a Phred score of 20 as minimum quality threshold, ends of all reads were trimmed accordingly using the FASTX-Toolkit 0.13 (available at http://hannonlab.cshl.edu/fastx_toolkit/, accessed Oct. 4, 2012). Mapping and methylation calling was then performed on all quality-corrected reads using the BS-methylation caller Bismark 0.7.3 (36) with the alignment tool Bowtie2 2.0 (37). Mapping options were standard, apart from an option for non-directional libraries, allowing a single mismatch in each seed region identified by Bowtie2 and permissive mismatch handling by allowing a decrease in 1.5 sequencing score points per base. Mapped reads were postprocessed by removing PCR duplicates (multiple, identical reads) using Samtools 0.1.18 (38). The mapping yield is shown in Table 2.

Annotation-specific results as shown in FIG. 2 were generated by a genome-wide survey of promoter-, gene-, repeat regions and CGIs based on the mapped position of CG sites.

Genome-wide promoter regions were obtained from the 2012 revision of the Eukaryotic Promoter Database (EPD), annotating a total of 9773 promoters (39), the current RepeatMasker annotation (Smit, A F A, Hubley, R & Green, P. RepeatMasker Open-3,0. 1996-2010, unpublished, available at www.repeatmasker.org, accessed Oct. 4, 2012) was used to determine repetitive elements, and the 2012 UCSC annotations for mm10 were used to discern CGIs and genes for mapped reads (40).

Verification of preprocessing and mapping accuracy using BLAST: To test for biases related to preprocessing and read mapping. BLAST+8 was used to perform local alignments of randomly sampled reads. In order to simulate methylation-aware mapping, C-to-T and G-to-A conversions were done on both query sequence and the mm10 reference genome and cross-aligned accordingly for both strands.

Average mapping efficiency from Bismark, which was 10.7% for single-cells, was compared with BLAST results and found to be as good or better. BLAST could map 6.8% of reads with the standard parameter settings (6.2% if only hits with a maximum of 4 mismatches was allowed.) The higher mapping yield of Bismark/Bowtie2 in comparison to BLAST could be due to Bismark's seed-match mapping strategy, the use of FASTQ-scores, and the ability to tolerate missing bases, shown in the sequence as N's4. For further investigation, some randomly chosen reads were BLASTed that were previously unmapped by both BLAST and Bismark against the nucleotide collection to find the closest match from mouse sequence or any contaminating DNA. It is concluded that low mapping efficiency results from lower sample quality and not from biases in the preprocessing or mapping. The comparably low yield using a local alignment also means that trimming and clipping of read borders cannot improve the mapping yield from our data further. To test for biases related to preprocessing and read mapping, BLAST+8 was used to perform local alignments of randomly sampled reads. In order to simulate methylation-aware mapping, C-to-T and G-to-A conversions were done on both query sequence and the reference genomes and cross-aligned accordingly for both strands. Average mapping efficiency from Bismark was compared with BLAST results and found to be as good or better, as follows: BLAST could map 6.8% of reads in this fashion and 6.2% if hits were limited by allowing a

maximum of 4 mismatches. The even lower mapping yield from BLAST, as compared to Bismark/Bowtie2, is attributed to its lack of a seed-match mapping strategy, lack of awareness of FASTQ-scores, inability to tolerate N's and BLAST's inexact matching algorithm. To investigate the potential origin of unmappable sequences, BLAST queries of some random previously unmapped sequences against the nucleotide collection were made to find the closest match. Some of the matches to our reads were bacteria such as Leptosphaeria, matches to human and ape DNA (suggesting reads with degenerated mouse sequence) and phage X174 (whose DNA was used for spike-in material). It was, therefore, demonstrated that low mapping efficiency stems from base-calling quality and is not due to biases in preprocessing or mapping options or software. The comparably low yield using a local alignment also means that trimming and clipping of read borders could not have been improved further to yield better results from the data.

### REFERENCES

1. Jones. P. A. DNA methylation and cancer. Oncogene 21, 5358-5360 (2002).
2. Laird, P. W. Principles and challenges of genomewide DNA methylation analysis. Nat Rev Genet 11, 191-203 (2010).
3. Gu, H. et al. Genome-scale DNA methylation mapping of clinical samples at single-nucleotide resolution, Nat Methods 7, 133-136 (2010).
4. Jeggo, P. A. & Holiday, R. Azacytidine-induced reactivation of a DNA repair gene in Chinese hamster ovary cells, Mol Cell Biol 6, 2944-2949 (1986).
5. Egger, G., Liang, G., Aparicio, A. & Jones, P. A. Epigenetics in human disease and prospects for epigenetic therapy. Nature 429, 457-463 (2004).
6. Frommer, M. et al. A genomic sequencing protocol that yields a positive display of 5-methylcytosine residues in individual DNA strands. Proc Natl Acad Sci USA 89, 1827-1831 (1992),
7. Khulan, B. et al. Comparative isoschizomer profiling of cytosine methylation: the HELP assay. Genome Res 16, 1046-1055 (2006).
8. Fraga, M. F. & Esteller, M. DNA methylation: a profile of methods and applications. Biotechniques 33, 632, 634, 636-649 (2002).
9. Raizis, A. M., Schmitt, F. & Jost, J. P. A bisulfite method of 5-methylcytosine mapping that minimizes template degradation. Anal Biochem 226, 161-166 (1995).
10. Genereux, D. P., Johnson, W. C., Burden, A. F., Stoger, R. & Laird, C. D. Errors in the bisulfite conversion of DNA: modulating inappropriate- and failed-conversion frequencies. Nucleic Acids Res 36, e150 (2008).
11. Hayes, J. D. et al. The Nrf2 transcription factor contributes both to the basal expression of glutathione S-transferases in mouse liver and to their induction by the chemopreventive synthetic antioxidants, butylated hydroxyanisole and ethoxyquin. Biochem Soc Trans 28, 33-41 (2000).
12. Loh, Y. H. et al. The Oct4 and Nanog transcription network regulates pluripotency in mouse embryonic stein cells. Nat Genet 38, 431-440 (2006).
13. Ushijima, T. et al. Fidelity of the methylation pattern and its variation in the genome. Genome Res 13, 868-874 (2003).
14. Laird, C. D. et al. Hairpin-bisulfite PCR: assessing epigenetic methylation patterns on complementary strands of individual DNA molecules. Proc Natl Acad Sci USA 101, 204-209 (2004).
15. Palii, S. S., Van Emburgh, B. O., Sankpal, U. T., Brown, K. D. & Robertson, K. D. DNA methylation inhibitor 5-Aza-2′-deoxycytidine induces reversible genome-wide DNA damage that is distinctly influenced by DNA methyltransferases 1 and 3B. Mol Cell Biol 28, 752-771 (2008).
16. Maslov, A. Y. et al. 5-Aza-2′-deoxycytidine-induced genome rearrangements are mediated by DNMT1. Oncogene (2012).
17. Ziller, M. J. et al. Genomic distribution and inter-sample variation of non-CpG methylation across human cell types. PLoS Genet 7, e1002389 (2011).
18. Faggioli, F., Sacco, M. G., Susani, L., Montagna, C. & Vezzoni, P. Cell fusion is a physiological process in mouse liver. Hepatology 48, 1655-1664 (2008).
19. Gundry, M., Li, W., Maqbool, S. B. & Vijg, J. Direct, genome-wide assessment of DNA mutations in single cells. Nucleic Acids Res 40, 2032-2040 (2012).
20. Good, J. M. Reduced representation methods for subgenomic enrichment and next-generation sequencing. Methods Mol Biol 772, 85-103 (2011).
21. Lister, R. et al. Human DNA methylomes at base resolution show widespread epigenomic differences. Nature 462, 315-322 (2009).
22. Schneider, E. et al. Spatial, temporal and interindividual epigenetic variation of functionally important DNA methylation patterns. Nucleic Acids Res 38, 3880-3890 (2011).
23. Heyn, H. et al. Distinct DNA methylomes of newborns and centenarians. Proc Natl Acad Sci USA 109, 10522-10527.
24. Busuttil, R. A. et al. Intra-organ variation in age-related mutation accumulation in the mouse. PLoS ONE 2, e876 (2007).
25. Schmitz, R. J. et al. Transgenerational epigenetic instability is a source of novel methylation variants. Science 334, 369-373.
26. Becker, C. et al. Spontaneous epigenetic variation in the Arabidopsis thaliana methylome. Nature 480, 245-249 (2011).
27. Rodriguez, J. et al. Genome-wide tracking of unmethylated DNA Alu repeats in normal and cancer cells. Nucleic Acids Res 36, 770-784 (2008).
28. Fraga, M. F. et al. Epigenetic differences arise during the lifetime of monozygotic twins. Proc Natl Acad Sci USA 102, 10604-10609 (2005).
29. Pantel, K. & Alix-Panabieres, C. Circulating tumour cells in cancer patients: challenges and perspectives. Trends Mol Med 16, 398-406 (2010).
30. Carver, T., Thomson, N., Bleasby, A., Berriman, M. & Parkhill, J. DNAPlotter: circular and linear interactive genome visualization. Bioinformatics 25, 119-120 (2009).
31. Garcia, A. M. et al. Detection and analysis of somatic mutations at a lacZ reporter locus in higher organisms: application to Mus musculus and Drosophila melanogaster. Methods Mol Biol 371, 267-287 (2007).
32. Tanaka K, Okamoto A (2007) Degradation of DNA by bisulfite treatment. Bioorg Med Chem Lett 17: 1912-1915.
33. Grunau C, Clark S J, Rosenthal A (2001) Bisulfite genomic sequencing: systematic investigation of critical experimental parameters. Nucleic Acids Res 29: E65-65.
34. Schmieder, R. & Edwards, R. Quality control and preprocessing of metagenomic datasets. Bioinformatics 27, 863-864 (2011).
35. Martin, M. Cutadapt removes adapter sequences from high-throughput sequencing reads. EMBnet.journal 17, pp. 10-12 (2011).
36. Krueger, F. & Andrews, S. R. Bismark: a flexible aligner and methylation caller for Bisulfte-Seq applications. Bioinformatics 27, 1571-1572 (2011).
37. Langmead, B. & Salzberg, S. L. Fast gapped-read alignment with Bowtie 2. Nat Methods 9, 357-359 (2012).
38. Heng L, H. B., Wysoker A, Fennell T. Ruan J, Homer N, Marth G, Abecasis G, and Durbin R. The Sequence Alignment/Map Format and SAMtools. Bioinformatics (Oxford, England) 25 2078^{⊥} 2079 (2009),
39. Perier, R. C., Praz, V., Junier, T., Bonnard, C. & Bucher, P. The eukaryotic promoter database (EPD). Nucleic Acids Res 28, 302-303 (2000).
40. Fujita, P. A. et al. The UCSC Genome Browser database: update 2011. Nucleic Acids Res 39, D876-882 (2010).

1. A method for determining a pattern of epimutations in a nucleic acid sequence comprising:
optionally, mixing the nucleic acid with an amount of a heterologous nucleic acid;
subjecting the nucleic acid to nucleic acid-denaturing conditions;
contacting the nucleic acid with sodium bisulfite at a temperature in excess of 50° C. so as to convert non-methylated cytosine residues of the nucleic acid into uracil residues;
recovering the converted nucleic acid;
subjecting the recovered converted nucleic acid to amplification with primers directed to the nucleic acid sequence;
sequencing the amplified product to determine the presence of uracil residues and cytosine residues, so as to thereby determine the pattern of epimutations in the nucleic acid sequence, wherein a cytosine residue in the amplified product indicates a methylated cytosine at the corresponding residue position in the nucleic acid sequence and wherein a uracil residue in the amplified product indicates a non-methylated cytosine at the corresponding residue position in the nucleic acid sequence.

2. The method of claim 1, further comprising comparing the epimutation pattern of the amplified product to a corresponding control sequence, so as to determine epimutations in the nucleic acid sequence relative to the control sequence.

3. The method of claim 1, wherein the nucleic acid is obtained from a cell.

4. The method of claim 1, further comprising obtaining the nucleic acid from a single cell.

5. The method of claim 1, wherein the heterologous nucleic acid is DNA or tRNA.

6. The method of claim 1, wherein the heterologous nucleic acid is a salmon sperm DNA or salmon sperm tRNA.

7. The method of claim 1, wherein the amplification primers are not directed to the heterologous nucleic acid.

8. (canceled)

9. The method of claim 1, wherein the nucleic acid is contacted with sodium bisulfite at a temperature of between 60° C. and 70° C. so as to convert methylated cytosine residues of the nucleic acid into uracil residues.

10. The method of claim 1, wherein the nucleic acid is contacted with sodium bisulfite at a temperature and for an amount of time sufficient as to convert 95% or more of the methylated cytosine residues of the nucleic acid into uracil residues.

11. The method of claim 1, further comprising contacting the nucleic acid, subsequent to the contact with sodium bisulfite, with carrier RNA.

12-14. (canceled)

15. The method of claim 1, wherein the method is performed on nucleic acid obtained from a single cell.

16-18. (canceled)

19. The method of claim 1, wherein the recovered nucleic acid is subjected to whole genome amplification using random hexamer primers.

20. The method of claim 1, wherein the amplification is multiple displacement amplification.

21-26. (canceled)

27. The method claim 1, wherein the recovered nucleic acid is subjected to nested PCR amplification prior to sequencing.

28. The method of claim 1, wherein recovering the converted nucleic acid comprises purifying the converted nucleic acid from remaining reagents and products using a nucleic acid purification column.

29. The method of claim 1, further comprising desulphonating the recovered nucleic acid.

30. A method for determining the effect of an agent on epimutation status of a nucleic acid comprising determining the epimutation pattern of a nucleic acid in the cells in a first portion of the cells, and contacting a second portion of the cells with the agent and determining epimutation pattern in a corresponding nucleic acid sequence in the second portion of the cells by the method of claim 1, and comparing the pattern of epimutations in the nucleic acid obtained from the first portion with the pattern of epimutations in the corresponding nucleic acid from the second portion so as to determine the effect of the agent on epimutation status of the cells.

31. (canceled)

32. A kit for performing the method of claim 1 comprising reagents as set forth in claim 1 and written instructions for use.

33. A system for performing the method of claim 1, comprising:
one or more data processing apparatus; and
a computer-readable medium coupled to the one or more data processing apparatus having instructions stored thereon which, when executed by the one or more data processing apparatus, cause the one or more data processing apparatus to perform the method of claim 1.

34. A computer-readable medium coupled to the one or more data processing apparatus having instructions stored thereon which, when executed by the one or more data processing apparatus, cause the one or more data processing apparatus to perform the method of claim 1.


SEQUENCE LISTING (29 sequences)

SEQ ID NO: 1 - DNA, 37 bp, Artificial Sequence
  Nfe212FE primer
aggaagagagtggtatagtttttagtttgtggagagt

SEQ ID NO: 2 - DNA, 51 bp, Artificial Sequence
  Nfe212RE primer
cagtaatacgactcactatagggagaaggctccacccaaaacccaacaaac

SEQ ID NO: 3 - DNA, 32 bp, Artificial Sequence
  Nfe212FI primer
aggaagagagagttatgaagtagtagtaaaaa

SEQ ID NO: 4 - DNA, 54 bp, Artificial Sequence
  nfe212RI primer
cagtaatacgactcactatagggagaaggctaatataatctcataaaaccccac

SEQ ID NO: 5 - DNA, 27 bp, Artificial Sequence
  Cyp71aFE primer
gagtgaatttttaagttatggttgttt

SEQ ID NO: 6 - DNA, 25 bp, Artificial Sequence
  Cyp71aRE primer
aacaaacaaaaactttccatcctaa

SEQ ID NO: 7 - DNA, 35 bp, Artificial Sequence
  Cyp71a FI primer
aggaagagagttgtttagaagatgagtgttgggag

SEQ ID NO: 8 - DNA, 57 bp, Artificial Sequence
  Cyp71aRI primer
cagtaatacgactcactatagggagaaggctcaaacaaaaactttccatcctaactt

SEQ ID NO: 9 - DNA, 35 bp, Artificial Sequence
  RabFE primer
aggaagagagtgattgggagttttgtagttttgta

SEQ ID NO: 10 - DNA, 59 bp, Artificial Sequence
  RabRE primer
cagtaatacgactcactatagggagaaggctaaaaaaaacaattactctaactttcaca

SEQ ID NO: 11 - DNA, 37 bp, Artificial Sequence
  RabFI primer
aggaagagaggtggtaaagtgtttgtttatagaaggg

SEQ ID NO: 12 - DNA, 56 bp, Artificial Sequence
  RabRI primer
cagtaatacgactcactatagggagaaggctaaataaacctccaaatctactaccc

SEQ ID NO: 13 - DNA, 25 bp, Artificial Sequence
  OCT4-reg2-FE primer
gttttggatatgggttgaaatattg

SEQ ID NO: 14 - DNA, 25 bp, Artificial Sequence
  OCT4-reg2-RE primer
ccaccctctaaccttaacctctaac

SEQ ID NO: 15 - DNA, 36 bp, Artificial Sequence
  OCT4-reg2-FI primer
aggaagagaggatatgggttgaaatattgggtttat

SEQ ID NO: 16 - DNA, 55 bp, Artificial Sequence
  OCT4-reg2-RI primer
cagtaatacgactcactatagggagaaggctaatcctctcacccctaccttaaat

SEQ ID NO: 17 - DNA, 34 bp, Artificial Sequence
  OCT4-reg1-FE primer
aggaagagaggaaggttgaaaatgaaggtttttt

SEQ ID NO: 18 - DNA, 56 bp, Artificial Sequence
  OCT4-reg1-RE&RI primer
cagtaatacgactcactatagggagaaggctccaccctctaaccttaacctctaac

SEQ ID NO: 19 - DNA, 33 bp, Artificial Sequence
  OCT4-reg1-FI primer
aggaagagagaaggttgaaaatgaaggtttttt

SEQ ID NO: 20 - DNA, 26 bp, Artificial Sequence
  L1chr18FE primer
tataggggaatgttagggttaagaag

SEQ ID NO: 21 - DNA, 26 bp, Artificial Sequence
  L1chr18RE primer
caaaacaataccacctcaaaactact

SEQ ID NO: 22 - DNA, 35 bp, Artificial Sequence
  L1chr18FI primer
aggaagagagataggggaatgttagggttaagaag

SEQ ID NO: 23 - DNA, 60 bp, Artificial Sequence
  L1chr18RI primer
cagtaatacgactcactatagggagaaggctcctaaaacctaaaaattacaaacataatt

SEQ ID NO: 24 - DNA, 25 bp, Artificial Sequence
  Dpf1FE primer
tttttgggatttagtttttgtttta

SEQ ID NO: 25 - DNA, 56 bp, Artificial Sequence
  Dpf1RE primer
cagtaatacgactcactatagggagaaggctacaaaaaccttttccttcaaatacc

SEQ ID NO: 26 - DNA, 35 bp, Artificial Sequence
  Dpf1FI primer
aggaagagagaggattagttgttgttttgtgatga

SEQ ID NO: 27 - DNA, 27 bp, Artificial Sequence
  Gabra1FE primer
tttttagggaaggtaagaaaagaggat

SEQ ID NO: 28 - DNA, 25 bp, Artificial Sequence
  Gagra1RE&RI primer
caacataaaaaacaaccaaacaaac

SEQ ID NO: 29 - DNA, 36 bp, Artificial Sequence
  Gabra1FI primer
aggaagagagtggagtttaaggtaaaggtatgtttt